(19) United States
(12) Patent Application Publication
(10) Pub. No.: US 2021/0338755 A1
(43) Pub. Date: Nov. 4, 2021

(54) AQUEOUS EXTRACT FROM CELLS OF FITZROYA CUPRESSOIDES (ALERCE) WITH ANTI- AGING AND SKIN REGENERATION PROPERTIES

(71) Applicants: RUBISCO BIOTECHNOLOGY, Santiago (CL); FUNDACION COPEC - UNIVERSIDAD CATÓLICA, Santiago (CL)

(72) Inventor: José Felipe AQUEA ZEBALLOS, Santiago (CL)

(21) Appl. No.: 17/283,438

(22) PCT Filed: Oct. 8, 2019
(86) PCT No.: PCT/IB2019/058568

§ 371 Date: Apr. 7, 2021

(30) Foreign Application Priority Data
Oct. 8, 2018 (CL) 2863-2018

(51) Int. Cl. A61K 36/14; A61K 8/9761; A61Q 19/08; A61P 35/00

(52) U.S. Cl. CPC A61K 36/14; A61K 8/9761; A61K 2236/53; A61P 35/00; A61K 2236/11; A61Q 19/08

(57) ABSTRACT

Provided are aqueous Alerce (Fitzroya cupressoides) cell extracts, which may be obtained from in vitro cultures of Alerce cells. The aqueous Alerce cell extracts promote cell regeneration, and therefore have anti-aging properties. The Alerce cell extracts have been found to have no toxic effects on human cells, and have been found to not boost skin cancer cells. Such extracts may be used in pharmaceutical or cosmetic formulations for skin care, especially to promote regeneration and/or anti-aging of the skin.

### CROSS-REFERENCE TO RELATED APPLICATION

This application claims priority to Chilean Patent Application No. 2863-2018, filed Oct. 8, 2018, which is incorporated herein by reference in its entirety.

### FIELD

The present disclosure relates generally to Alerce extracts, and more specifically to Alerce cell extracts that have cell regeneration and anti-aging properties, and are suitable for use in pharmaceutical and/or cosmetic compositions for skin care.

### BACKGROUND

Fitzroya is a genus of plants belonging to the family of Cupresaceae. It is a monotypic genus, whose only species is Fitzroya cupressoides, a huge tree known as Alerce or Patagonian Alerce in their countries of origin: Argentina and Chile. This tree is a slow growing species, and also very long-lived. It has been reported that a specimen known as the “Great Grandfather” in the Alerce Coastal National Park in Chile is over 3622 years old, which makes it the third oldest living tree in the world.

Its wood is light, of an intense reddish color, and resistant to rot and the attack of insects. It can split very well following the grain, which allows the wood to be split with an axe obtaining long, thin boards. For these reasons, this wood is highly valued. At the beginning of the 20th century, an intense deforestation put this tree population at high risk of extinction. Currently. Fitzroya cupressoides is under strict protection program, so its exploitation is prohibited in Argentina and Chile.

There are a few studies published on the physiology or metabolism of Fitzroya cupressoides. For example, there has been one study on the sesquiterpenes of tree foliage. See Cool, L. G., Sesquiterpene alcohols from foliage of Fitzroya cupressoides, Phytochemistry, 42(4), 1015-1019 (1996). In this study, no use of Alerce leaves was determined. The study provided a description of some phytochemical components, but not their possible functions or applications. In another example, U.S. Pat. No. 5,955,621, Alerce seed and sprout cells were used to evaluate the presence of taxane compounds (for antineoplastic use) in plant cells.

Thus, what is needed in the art are commercially viable methods to obtain useful compositions from Alerce.

### BRIEF SUMMARY

The present disclosure provides an aqueous extract obtained from Alerce cells, which has pharmaceutical and cosmetic properties, particularly in skin regeneration and anti-aging effect. This extract is useful in developing products for the treatment and protection of the skin in order to promote health, and reduce the appearance of wrinkles and expression lines, as well as tissue regeneration in cases of recent injury or scarring.

In some aspects, provided herein are methods for in vitro culturing of Alerce cells; and obtaining an Alerce cell extract from the cell culture. In some embodiments, provided is a process for obtaining the extract in water that is friendly to the environment, as well as to the skin on which the resulting compositions will be applied. In some variations, in vitro cultures of Alerce cells were obtained by subjecting the cells to mechanical lysis in aqueous solvent, following by centrifugation to isolate the supernatant that includes the Alerce cell extract.

The aqueous Alerce cell extracts promote cell regeneration, and therefore have anti-aging properties, using the standard scratch test on keratinocytes. In some variations, the Alerce cell extracts stimulate up to 120% the regeneration of keratinocytes over the basal condition (control water). Additionally, the possible toxicity of the Alerce cell extracts was evaluated, for which the effect of the extract on the viability of HaCaT cells was studied. The Alerce cell extracts were found to have no toxic effects on human cells. It was also determined that the Alerce cell extracts do not boost skin cancer cells, when evaluating the effect of the extract on C32 melanoma cells.

In accordance with the above, the Alerce cell extracts have been found to suitable for use in pharmaceutical or cosmetic formulations for skin care, especially to promote regeneration and/or anti-aging of the skin.

### DESCRIPTION OF THE FIGURES

The present application can be understood by reference to the following description taken in conjunction with the accompanying figures.

FIG. 1. Feasibility test of the keratinocyte cell line called HaCaT after being exposed for 24 hours to Alerce extract 1, at different dilutions between 1 (extract as obtained in the example) and serial dilutions of this from 0.125% w/v at 0.002% w/v. As a control, water and 10% bovine fetal serum (10% FBS) were used, which are known to be non-toxic to the cells. The results show that the extract did not affect cell viability under any of the conditions tested.

FIG. 2. Scratch test, repair percentage of a scratch of a monolayer of epidermal cells at 24 hours of incubation with culture medium supplemented with water (negative control), 10% FBS (positive control), 10 ng/mL EGF (positive control), and extracts obtained in example 1 at dilutions 0.1250, 0.0313, 0.0078, 0.0020 and 0.0005% w/v. The extracts were observed to have better results than water control at all concentrations tested.

FIG. 3. Scratch test, repair percentage of a scratch of a monolayer of epidermal cells at 48 hours of incubation with culture medium supplemented with water (negative control), 10% FBS (positive control), 10 ng/mL EGF (positive control), and extracts obtained in example 1 at dilutions 0.1250, 0.0313, 0.0078, 0.0020 and 0.0005% w/v. The extracts were observed to have better results than water control at all concentrations tested.

FIG. 4. Viability test of the C-32 melanoma cell line after being exposed for 24 hours to Alerce extract 1, at different dilutions between 1 (extract as obtained in the example) and serial dilutions of this from 0.125% w/v to 0.002% w/v. As a control, water and 10% bovine fetal serum (10% FBS) were used, which are known to be non-toxic to the cells. The results show that the extract affected cell viability at the highest concentrations tested.

FIG. 5. Evaluation of the effect of mitomycin C in wound healing repair. The concentrations used are 10% fetal bovine serum (FBS), 10 ng/mL epithelial growth factor (EGF), and 0.125% Alerce extracts. 20 μmol/L of Mitomycin C was applied before each treatment. Different letters show significant differences. P<0.05

### DETAILED DESCRIPTION

The following description sets forth exemplary methods, parameters and the like. It should be recognized, however, that such description is not intended as a limitation on the scope of the present disclosure but is instead provided as a description of exemplary embodiments.

In some aspects, provided is an aqueous plant extract of Alerce (Fitzroya cupressoides) cells with properties for cellular regeneration and anti-aging of skin.

In some embodiments, the extract comprises one or more of the following main compounds: 4,5-dihydroxyphthalate: 2″,4″,6″-triacetylglycine: 6-phosphogluconic acid; 2-O-cafeoylhydroxycitric acid; catechin 7-O-beta-D-xyloside; 4,5-di-O-cafeoylquinic acid; 5,7,2′,3′,4′-pentahydroxy-3,6-dimethoxylavone-7-glucoside; diacylglycerol substituted with two unsaturated fatty acids of 19 and 12 carbons (also referred to herein as “PE (19:0/12:0)”); myricetin 3,7-diglucuronide; and torosaflavone.

In some variations, the extract comprises the following main compounds in the amounts (weight percentage of the total composition) indicated:

4,5-dihydroxyphthalate (0.4%-6.1%),

2″,4″,6″-triacetylglycine (0.25%-6.03%),

6-phosphogluconic acid (0.43%-4.32%),

2-O-cafeoylhydroxycitric acid (0.34%-3.86%),

catechin 7-O-beta-D-xyloside (0.37%-3.23%),

4,5-di-O-cafeoylquinic acid (0.26%-3.09%),

5,7,2′,3′,4′-pentahydroxy-3,6-dimethoxylavone-7-glucoside (0.25%-2.68%)

PE (19:0/12:0) (0.3%-2.09%),

myricetin 3,7-diglucuronide (0.7%-1.29%), and

torosaflavone (0.19%-1.26%).

In other aspects, provided is a process for obtaining the extracts described herein. In some embodiments, the process comprises: a) mixing Alerce cells with water to a concentration between 0.0005% w/v to 10% w/v; b) incubating the mixture while stirring at a temperature between 10° C. to 40° C. for a period between 1 hour and 5 days; and c) separating the incubated mixture to obtain an aqueous phase, wherein the aqueous phase comprises the extract.

In one embodiment, the process comprises: a) gradually mixing Alerce cells with water to a concentration between 0.0005% w/v to 10% w/v; b) incubating the mixture while stirring from 100 to 250 rpm at a temperature between 10° C. to 40° C. for a period between 1 hour and 5 days; and c) separating the mixture to obtain an aqueous phase, wherein the aqueous phase comprises the extract.

In some variations of the foregoing, the process further comprise some or all of the following steps: sterilizing the extract; and/or storing the extract at temperatures between 4° C. and −80° C.; and/or freeze drying the extract.

In some embodiments, the Alerce cells were mixed with water in step a) to a concentration between 0.001% w/v to 5% w/v, or between 0.05% to 3%. In some variations, the water is sterile. In certain variations, the water is sterile and filtered. In one variation, the water is filtered with a 0.22 μm nitrocellulose filter.

In some embodiments, when the incubation of the mixture in step b) is performed at a higher temperature, less extraction time is required. For example, in one variation, the incubation temperature is 40° C., and the incubation period is between 1 hour to 12 hours. In another variation, the incubation temperature is 30° C., and the incubation period is between 1 hour to 24 hours. In another variation, the incubation temperature is 20° C., and the incubation period is between 12 hours to 4 days. In yet another variation, the incubation temperature is 10° C., and the incubation period is between 1 to 5 days.

In some embodiments, step c) comprises separating the aqueous phase, which includes the extract, and discarding the pellet or precipitate. This separation can be carried out by any suitable methods or techniques known in the art, including, for example, centrifugation and filtration. In one variation, the separation is carried out by centrifugation for 5 to 30 minutes at a speed of between 4000 to 12000 rpm.

In certain embodiments where the process includes sterilization of the extract, the sterilization is carried out, for example, by filtration with a 0.22 μm filter, or by means of UV radiation, or by any other method or technique known in the art that does not alter the composition or stability of the extract.

In other aspects, provided is the use of the Alerce cell extracts described herein for cell regeneration and anti-aging in pharmaceutical and/or cosmetic compositions for skin care. The Alerce cell extracts described herein show excellent properties in tissue regeneration, and therefore anti-aging effects. The effects of the extracts on melanomas, specifically on skin cancer cells, were studied. The studies showed that the Alerce cell extracts described herein negatively affected the cell viability of cancer cells, and did not favor cell proliferation. In this way, the Alerce cell extracts can be used without problems on the skin, so as to favor the regeneration of the skin or promote its anti-aging. In cases where the skin has a melanoma, the growth of the carcinogenic cells are enhanced by the Alerce cell extracts. Rather, the Alerce cell extracts may even inhibit growth of the carcinogenic cells.

The Alerce cell extracts described herein are obtained from an in vitro culture of Alerce cells and the processes described herein present several advantages. The processes do not require the harvest of raw materials, do not harm the species, and the extracts can be obtained at will without reliance on the seasons (in contrast to many plant extracts that require the harvest of the raw material). Additionally, this ensures the reproducibility of the extract in its constituents, since the environmental conditions are controlled, and the biomass is homogeneous.

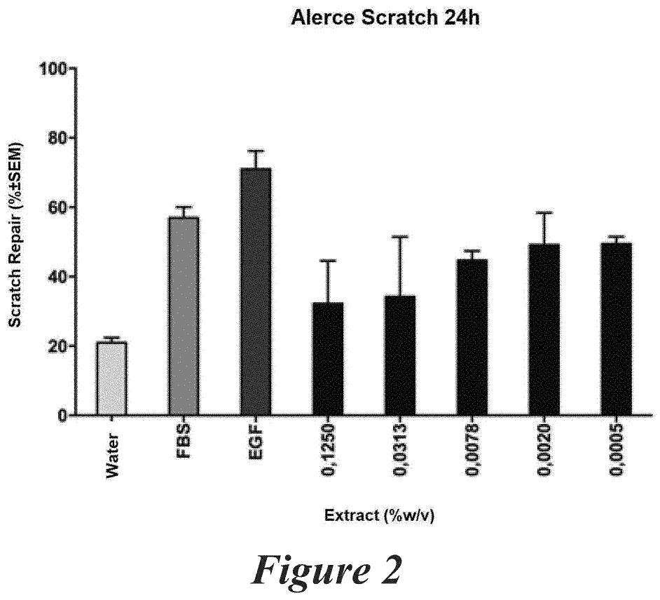

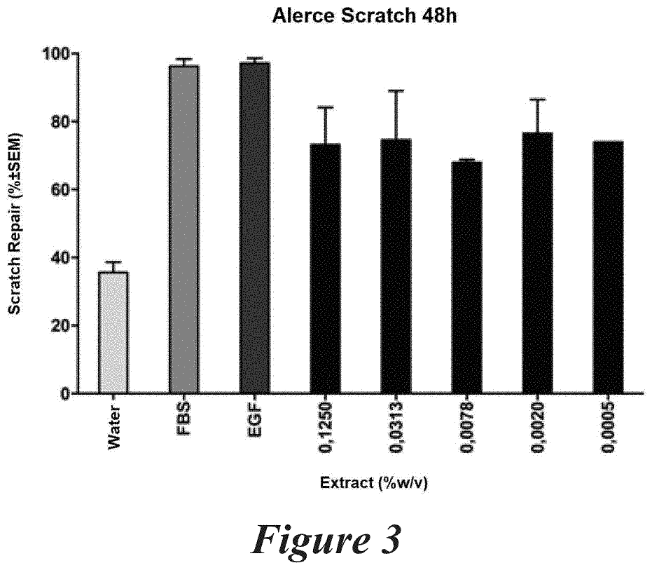

The Alerce cell extracts described herein have demonstrated excellent properties for use on human skin, and do not present any degree of toxicity or negative effect on keratinocytes (HaCaT cell line). Additionally, Alerce cell extracts described herein promote cell regeneration, using the scratch test (FIGS. 2 and 3), where an injury to a monolayer of epidermal cells is performed and the percentage of repair of said lesion is measured in a given time, the extracts resulted in a repair rate that was twice compared to the water control at 24 and 48 hours, reaching a repair percentage of around 70 to 80% of the injury at 48 hours. These results allow confirmation that the extract has an effect on tissue regeneration, and therefore an anti-aging effect and or repair or prevention of wrinkles or expression lines, and on regeneration in wounds and scars.

A fundamental aspect of the products to be used on the skin is the activity that they present on skin cancer cells, or melanoma, particularly since in cases where products promote their proliferation, such products cannot be used on the skin. In this case, the Alerce cell extracts described herein were tested on melanoma cell lines, and not only did not stimulate growth or proliferation, but even inhibited it by up to 20%, which indicates a safe extract for pharmaceutical and/or cosmetic use. In this way, the Alerce cell extracts described herein can be safely used in preparations intended for the care or treatment of the skin, without negative effects and promoting the health thereof.

In some embodiments, the Alerce cell extracts described herein can be used in pharmaceutical formulations, such as creams, serums or ointments to promote the regeneration of the skin; as well as in cosmetic formulations to obtain anti-aging effects or prevention, repair or reduction of wrinkles and expression lines. In some variations, the formulations comprising the Alerce cell extracts described herein are creams, serum, gels, cleansers, or lotions. In some variations, the formulations further comprise other ingredients, such as glycerin, carriers, perfumes, preservatives, dyes, or other ingredients that do not modify the properties of the Alerce cell extracts described herein.

In other embodiments, the pharmaceutical and/or cosmetic formulations further comprises other active compounds or compounds with properties complementary to those of the Alerce cell extracts described herein. For example, in some variations, the formulation further comprise vitamins (e.g., vitamin E), minerals or others ingredients that can achieve synergistic benefits. In one variation, the cell extracts described herein may be incorporated into a sunscreen formulation.

The pharmaceutical and/or cosmetic compositions may be formulated with the Alerce cell extracts described herein by incorporating a volume of the extract obtained by the processes herein. For example, in some variations, a concentration between 0.0005 to 1% w/v of the Alerce cell extracts is formulated to achieve a final product (to be used directly on the skin) having a final concentration between 0.03% to 1% w/v of the extract.

In other aspects, provided is an article of manufacture, such as a container comprising the Alerce cell extracts described herein; and a label containing instructions for use of such extracts. In yet other aspects, provided is a kit comprising the Alerce cell extracts described herein; and a package insert containing instructions for use of such extracts. In some variations of the foregoing, suitable uses include skin regeneration, reducing aging skin effect, or preventing or avoiding wrinkles or expression lines. In one variation, the Alerce cell extracts described herein may be used on the skin of a human having melanoma, and the use of the extracts may decrease viability of melanoma cells.

### Enumerated Embodiments

The following enumerated embodiments are representative of some aspects of the invention.

1. A composition for cell regeneration and anti-aging CHARACTERIZED in that it comprises aqueous extracts of Alerce cells (Fitzroya cupressoides) cultured in vitro.
2. The composition according to embodiment 1 CHARACTERIZED in that in addition to the extract of Alerce cells cultured in vitro, it comprises active compounds or compounds with properties complementary to those of the extract of the invention, such as vitamins, sunscreens, minerals or others.
3. The composition according to embodiment 1 CHARACTERIZED in that the major compounds comprising the extract correspond to 4,5-Dihydroxyphthalate (0.4%-6.1%), 2″,4″,6″-Triacetylglycine (0.25%-6.03%), 6-Phosphogluconic acid (0.43%-4.32%), 2-O-Cafeoylhydroxycitric acid (0.34%-3.86%), Catechin 7-O-beta-D-xyloside (0.37%-3.23%), 4,5-Di-O-cafeoylquinic acid (0.26%-3.09%), 5,7,2′,3′,4′-Pentahydroxy-3,6-dimethoxyfllavone 7-glycoside (0.25%-2.68%), PE (19:0/12:0) (0.3%-2.09%), Myricetin 3,7-diglucuronide (0.7%-1.29%) and Torosaflavone (0.19%-1.26%).
4. Process for obtaining the extracts from cells cultured in vitro according to embodiment 1 CHARACTERIZED in that it comprises the following steps:
a) mixing the Alerce cells with water little by little up to a concentration between 0.05% w/v and 10% w/v;
b) incubating the mixture while stirring from 100 to 250 rpm at a temperature between 10 to 40° C. for a period between 1 hour and 5 days; and
c) separating the aqueous phase, which constitutes the extract.
5. Process of obtaining according to embodiment 4 CHARACTERIZED in that it optionally comprises some or all of the following steps:
d) sterilizing the extract obtained;
e) storing cold at temperatures between 4° C. and −80° C.
f) freeze drying.
6. Process of obtaining according to any of embodiments 4 or 5 CHARACTERIZED in that in step a) the concentration of the suspension is between 0.1% w/v and 5% w/v.
7. Process of obtaining according to embodiments 4 or 5 CHARACTERIZED in that in step b) is incubated at 40° C. for between 1 hour and 12 hours.
8. Process of obtaining according to embodiments 4 or 5 CHARACTERIZED in that in step b) is incubated at 30° C. for between 1 hora and 24 horas.
9. Process of obtaining according to embodiments 4 or 5 CHARACTERIZED in that in step b) is incubated at 20° C. for between 12 horas and 4 dias.
10. Process of obtaining according to embodiments 4 or 5 CHARACTERIZED in that in step b) is incubated at 10° C. for between 1 and 5 dias.
11. Process of obtaining according to embodiments 4 or 5 CHARACTERIZED in that in step c) separation can be done by centrifugation or filtration.
12. Process of obtaining according to embodiment 5 CHARACTERIZED in that in step d) the sterilization can be done by filtration with a 0.22 μm filter, or by UV radiation.
13. Use of an extract according to any of embodiments 1 to 3 CHARACTERIZED in that it is used to obtain formulations for pharmaceutical or cosmetic skin care use.
14. Use according to embodiment 13 CHARACTERIZED in that the extract is used to prepare a medicine for skin regeneration.
15. Use according to embodiment 13 CHARACTERIZED in that the extract is used to prepare a composition with anti-aging skin effect.
16. Use according to embodiment 13 CHARACTERIZED in that the extract is used to prepare a composition to prevent or avoid wrinkles or expression lines.
17. Use according to embodiment 13 CHARACTERIZED in that the extract is used to prepare a composition to decrease the viability of melanoma cells.
18. Composition for skin care CHARACTERIZED in that it comprises formulation excipients and the composition of embodiments 1 to 3, in a final concentration of between 0.0005% w/v and 10% w/v.
19. Composition for skin care according to embodiment 18 CHARACTERIZED in that it comprises formulation excipients and the composition of embodiments 1 to 3, in a final concentration of between 0.0005% w/v and 5% w/v.
20. Composition for skin care according to embodiment 19 CHARACTERIZED in that it comprises formulation excipients and the composition of embodiments 1 to 3, in a final concentration of between 0.05% w/v and 1% w/v.

### EXAMPLES

The presently disclosed subject matter will be better understood by reference to the following Examples, which are provided as exemplary of the invention, and not by way of limitation.

### Example 1: Preparation of Alerce Cell Extracts

Extracts can be made in several ways, varying the time, cost and equipment used in their preparation. One method for obtaining extracts of the invention is detailed below.

0.2 g of fresh Alerce cells was taken and washed to remove traces of the culture medium, and deposited in a flask, 20 ml of water was added little by little, obtaining a 1% weight/volume suspension. It was then incubated while stirring at 180 rpm, at a temperature of 30° C., for 2 hours. Then, it was allowed to cool to room temperature at rest, the entire content was passed to a 50 mL Falcon tube which was centrifuged for 10 minutes at 8000 rpm. The supernatant was removed, discarding the solid tissue, thus obtaining the extract. Additionally, the extract obtained was sterilized by filtration with a 0.22 μm filter, and part was stored at −20° C. and part at −80° C.

The obtained extract was analyzed in its composition by the high performance liquid chromatography method (or HPLC) and the majority constituents were determined to be: 4,5-dihydroxyphthalate (6.1%), 2″,4″,6″-triacetylglycine (6.03%), 6-phosphogluconic acid (4.32%), 2-O-cafeoylhydroxycitric acid (3.86%), catechin 7-O-beta-D-xyloside (3.23%), 4,5-di-O-caffeoquinine acid (3.09%), 5,7,2′,3′,4′-pentahydroxy-3,6-dimethoxyflavone 7-glycoside (2.68%) PE (19:0/12:0) (2.09%), myricetin 3,7-diglucuronide (1.29%) and torosaflavone (1.26%). It should be understood that PE (19:0/12:0) is diacylglycerol, a derivative of phosphatidylethanolamine, substituted with two unsaturated fatty acids of 19 and 12 carbons respectively.

### Example 2: Toxicity Evaluation on Human Skin Cells

In this example, toxicity of the Alerce extracts obtained in Example 1 on skin was evaluated. This Example 2 was performed on human skin cells. The chosen model is the keratinocyte cell line called HaCaT, which represents the human epidermis (the outermost layer of skin).

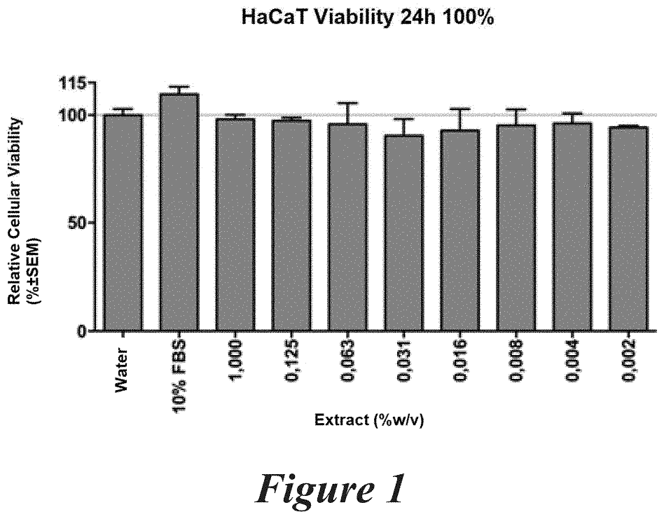

A viability test was carried out, with which the metabolic activity of the cells was evaluated after being exposed for 24 hours to Alerce extract 1, which was diluted at different concentrations between 1% (extract as obtained in the example) and serial dilutions of this from 0.125% w/v to 0.002% w/v. All concentrations are expressed in % w/v that considers initial cell weight by final volume. Additionally, 10% bovine fetal serum (10% FBS) and water (without FBS) were tested as controls, which are known to be non-toxic to the cells, maintaining 100% viability. This result shown in FIG. 1 provide proof where it can be seen that none of the dilutions studied affects cell viability, which allows the conclusion that the Alerce extract is not toxic to skin cells.

### Example 3: Evaluation of the Effect on Tissue Regeneration

This Example describes a second functional test, called a scratch test, which is based on performing an injury on a monolayer of epidermal cells. The repair area of the lesion is evaluated by incubating the wounded epidermal cells in the presence of different compounds added to the culture medium, whether that is the controls, or the extract of interest, or in this case different concentrations of Alerce extracts compared to a positive control. The results were analyzed according to the area of the initial lesion and how this area was reduced over time. At the end, a calculation was made to obtain the percentage of repair of the lesion.

The following medium compositions were used as controls: 1) Negative Control: poor culture medium without supplementation or control Water (without Fetal Bovine Serum, FBS), 2) 10% FBS: rich culture medium with 10% FBS, and 3) EGF 10 ng/mL: poor culture medium supplemented with 10 ng/mL Epidermal Growth Factor.

The extracts obtained in Example 1, which were used at dilutions 0.1250, 0.0313, 0.0078, 0.0020 and 0.0005% w/v on the culture medium were evaluated. The percentage of scratch repair was evaluated first at 24 hours of incubation (the results of this test are shown in FIG. 2), and at 48 hours of incubation (the results of this test are shown in FIG. 3).

After 24 hours of treatment, the results show that the extracts have better results than the water control in all the concentrations tested. After 48 hours of treatment, the extracts at 0.125% w/v achieve more than 70% of scratch repair, compared with 38% repair in the water control. Again, the extracts have better results than water control at all concentrations tested.

These results demonstrate that the extracts actively promote cell regeneration, which is directly related to an effect on tissue regeneration, anti-aging and the repair or prevention of wrinkles or expression lines, and on regeneration in wounds and scars.

### Example 4: Evaluation of Effect on Melanomas

In order to explore whether the extracts of the invention can effectively be used safely on human skin, cell viability tests were conducted, this time focusing on another skin model, melanoma, by means of the cell line C-32. This model is characterized as being a tumor, presenting characteristics of being immortal, and a high proliferative capacity.

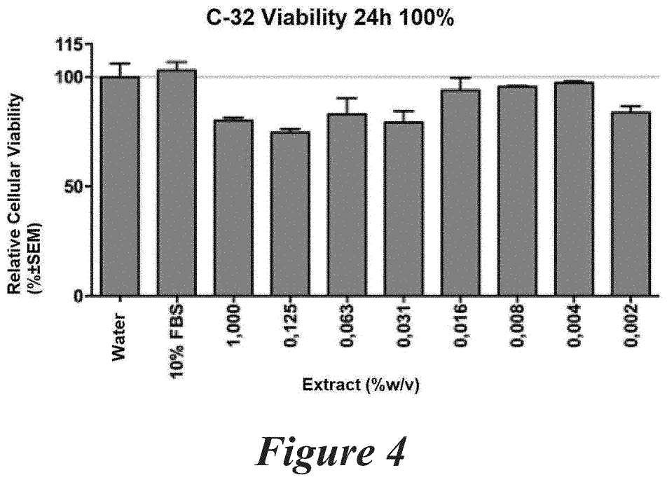

A feasibility test was carried out and the metabolic activity of C-32 cells was evaluated after being exposed for 24 hours to the Alerce extract obtained in example 1, which was diluted at different concentrations between 1% (extract which was obtained in the example) and serial dilutions from 0.125% w/v to 0.002% w/v. Additionally, 10% fetal bovine serum (10% FBS) and water (without FBS) were incorporated as controls. The results of the viability test of the melanoma model, versus the treatment with the extract can be seen in FIG. 4. The results show that the Alerce extract at the highest concentrations tested (between 1% and 0.031%) affects the viability of the melanoma cell line, reaching between 75% and 80% viability.

Thus, in case the skin to be treated with the extract had a melanoma, the use of the extracts could even be beneficial. It was found here that the cancer cells would not be stimulated by the Alerce extracts.

### Example 5: Cosmetic or Pharmaceutical Skin Care Composition

The Alerce extract obtained in Example 1 was incorporated into a glycerin-based cream at a final concentration 0.03% w/v, which can be used directly on the skin.

The preceding examples demonstrate the beneficial properties of the Alerce extracts described herein for the skin. Modifications to these embodiments will be apparent to those skilled in the art, which will maintain the properties of the extracts, all these modifications falling within the scope of the invention, as indicated in the appended claims.

### Example 6: Study of Effect of Mitomycin C in Wound Healing Repair

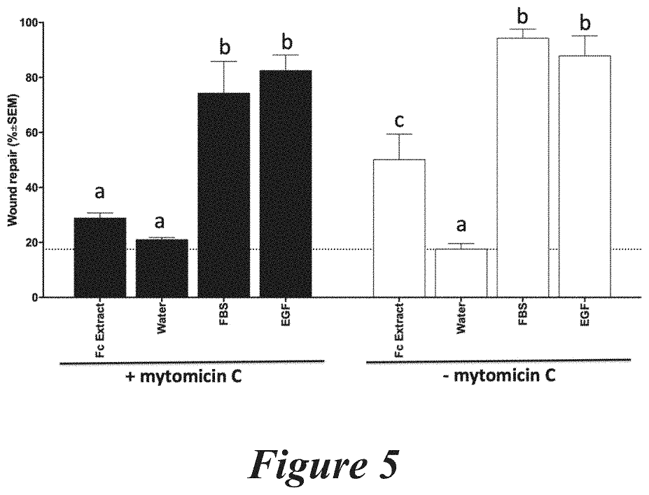

This Example focuses on the isolation of the different processes involved in wound healing in order to identify the specific mechanism that explained the beneficial effects of the Alerce extract. Scratch wound experiments at 48 hours using cell division inhibitors provided insight into the mechanism of Alerce cell culture extract's positive effect on wound closure. The application of mitomycin inhibits division of the HaCaT cells that, together with cell migration, are the two mechanisms involved in wound healing repair. The use of mitomycin C significantly inhibited the wound closure induced by a 0.125% v/v of Alerce cell culture extract at 48 hours (FIG. 5). In this Example, Alerce cell culture extracts accelerated the healing process up to 190% in absence of mytomicin C, which was reduced to 33% in presence of the cell division inhibitor relative to basal cell line conditions. This result suggested that Alerce cell culture extract accelerated wound healing by stimulating keratinocyte cell division.

1. A composition for cell regeneration and anti-aging, comprising an aqueous extract of Alerce cells (Fitzroya cupressoides) cultured in vitro.

2. The composition of claim 1, wherein the aqueous extract comprises: 4,5-dihydroxyphthalate, 2″,4″,6″-triacetylglycine, 6-phosphogluconic acid, 2-O-cafeoylhydroxycitric acid, catechin 7-O-beta-D-xyloside, 4,5-di-O-cafeoylquinic acid, 5,7,2′,3′,4′-pentahydroxy-3,6-dimethoxyflavone 7-glycoside, diacylglycerol substituted with two unsaturated fatty acids of 19 and 12 carbons, myricetin 3,7-diglucuronide, and torosaflavone.

3. The composition of claim 1, wherein the composition is formulated for pharmaceutical or cosmetic skin care use.

4. The composition of claim 1, further comprising at least one skin care excipient.

5. The composition of claim 1, further comprising vitamins, sunscreen actives, or minerals, or any combination thereof.

6. The composition of claim 1, wherein the extract is present in the composition in a final concentration of between 0.0005% w/v and 10% w/v.

7. The composition of any claim 1, wherein the extract is present in the composition in a final concentration of between 0.0005% w/v and 5% w/v.

8. The composition of claim 1, wherein the extract is present in the composition in a final concentration of between 0.05% w/v and 1% w/v.

9. A process for obtaining extracts from Alerce cells (Fitzroya cupressoides) cultured in vitro, comprising; a) mixing Alerce cells with water to a concentration between 0.05% w/v and 10% w/v; b) incubating the mixture at a temperature between 10 to 40° C. for a period between 1 hour and 5 days; and c) separating the mixture to obtain an aqueous phase comprising the extract.

10. The process of claim 9, further comprising: sterilizing the extract; or storing the extract at temperatures between 4° C. and −80° C.; or freeze drying the extract, or any combination of thereof.

11. The process of claim 9, wherein in step a), the concentration is between 0.1% w/v and 5% w/v.

12. The process of claim 9, wherein in step b), the incubation is performed at 40 CC for a period between 1 hour and 12 hours.

13. The process of claim 9, wherein in step b), the incubation is performed at 30° C. for a period between 1 hora and 24 hours.

14. The process of claim 9, wherein in step b), the incubation is performed at 20° C. for a period between 12 horas and 4 days.

15. The process of claim 9, wherein in step b), the incubation is performed at 10° C. for a period between 1 and 5 days.

16. The process of claim 9, wherein in step c), the separation is performed by centrifugation or filtration.

17. The process of claim 9, wherein in step d), the sterilization is performed by filtration with a 0.22 pm filter, or by UV radiation.

18. A composition produced according to the process of claim 9.

19. A method for treating a human in need thereof, comprising: applying a composition of claim 1 to skin of the human for pharmaceutical or cosmetic use.

20. The method of claim 19, wherein the treating comprises skin regeneration; reducing aging skin effect; or preventing or avoiding wrinkles or expression lines.

21. The method of claim 19, wherein the human has melanoma, and wherein the treating further comprises decreasing viability of melanoma cells.
